(19) United States
(12) Patent Application Publication
(10) Pub. No.: US 2009/0130175 A1
(43) Pub. Date: May 21, 2009

(54) Composite Injectable and Pre-Fabricated Bone Replacement Material and Method for the Production of Such Bone Replacement Material

(75) Inventors: Michael Liebschner, Pearland, TX (US); Peter L. Schuerman, Houston, TX (US)

Correspondence Address: CONLEY ROSE, P.C., 5601 GRANITE PARKWAY, SUITE 750, PLANO, TX, 75024, US

(73) Assignee: William Marsh Rice University, Houston, TX (US)

(21) Appl. No.: 12/358,456

(22) Filed: Jan. 23, 2009

Related U.S. Application Data
(62) Division of application No. 10/549,415, filed on Oct. 20, 2006.
(60) Provisional application No. 60/454,485, filed on Mar. 13, 2003.

(51) Int. Cl. A61F 2/28

(52) U.S. Cl. 424/426

(57) ABSTRACT

A bone replacement material and methods for making and using the same are disclosed. In one embodiment, the bone replacement material comprises a viscous component. The bone replacement material also comprises a plurality of biodegradable inclusions, wherein the inclusions comprise polymers. The inclusions can comprise polymers with therapeutic agents. In other embodiments, the inclusions have a high surface to volume ratio.

### CROSS-REFERENCE TO RELATED APPLICATIONS

This is a Divisional Application of U.S. patent application Ser. No. 10/549,415, filed Oct. 20, 2006 and published as US 2007/0065652 A1, and entitled “Composite Injectable and Pre-Fabricated Bone Replacement Material and Method for the Production of Such Bone Replacement Material,” which was a filing under 35 U.S.C. 371 of International Application No. PCT/US2004/007600 filed Mar. 12, 2004, entitled “Composite Injectable and Pre-Fabricated Bone Replacement Material and Method for the Production of Such Bone Replacement Material,” claiming priority of U.S. Provisional Patent Application 60/454,485 filed Mar. 13, 2003, which applications are incorporated by reference herein in their entirety.

### STATEMENT REGARDING FEDERALLY SPONSORED RESEARCH OR DEVELOPMENT

The research was sponsored through the Texas Higher Education Coordinating Board (Austin, Tex.) with an Advanced Technology Program grant #003604-0031-2001.

### FIELD OF THE INVENTION

The present invention generally relates to the field of medical devices and more specifically to using material comprising a composite material and biodegradable inclusions to replace bone.

### BACKGROUND OF THE INVENTION

Bone replacement materials are of increasing importance in the orthopedic, cranio-maxillofacial, and dental fields. Materials that set into solid, calcium-containing mineral products are of particular interest as such products can closely resemble the mineral phase of natural bone and are potentially remodelable. The bone replacement materials are used for repairing fractured bone, strengthening cancerous bone, reinforcing osteoporotic bone, accelerated dental implant anchorage, and the like.

Such bone replacement materials typically comprise bone cement. The cements typically form a flowable, paste-like material that is capable of setting into a solid product after being injected into bone. Different formulations of bone cement have been developed. One such typical formulation is the polymer polymethylmethacrylate (PMMA). However, natural bone mainly comprises a calcium phosphate having hydroxyapatite with which PMMA has poor biocompatibility. PMMA is biologically inert and does not actively promote tissue adhesion or formation on its surface. Therefore, it is preferable for bone replacement materials to be osteoconductive and thereby related chemically with natural bone. Bone cements including calcium carbonate and calcium phosphate have been developed to improve biocompatibility with natural bone. Drawbacks to such calcium-containing cements include slow biodegradation of the cement. Biodegradation of the cement is desired for the regeneration and growth of bone. The calcium-containing cements are typically slow-absorbable and thereby have poor biodegradation qualities.

Consequently, there is a need for a bone replacement material having improved biocompatibility with natural bone. Further needs include a bone replacement material that facilitates the regeneration and growth of bone. Additional needs include a bone replacement material that is biodegradable.

### SUMMARY OF THE INVENTION

These and other needs in the art are addressed in one embodiment by a material having an interconnected pore structure. The material comprises a viscous component, preferably made of one type of polymer. In addition, the material has a plurality of biodegradable inclusions, wherein the inclusions comprise polymers.

In another embodiment, the present invention includes a bone replacement material comprising a viscous component. The bone replacement material also comprises a plurality of biodegradable inclusions, wherein the inclusions comprise polymers.

A further embodiment of the present invention includes a method for creating a bone replacement material, wherein the bone replacement material comprises a composite material. The method comprises providing a viscous component and a plurality of biodegradable inclusions, wherein the inclusions comprise polymers. In addition, the method comprises combining the viscous component and the plurality of inclusions to produce the bone replacement material. The bone replacement material is preferably produced prior to implantation.

An additional embodiment of the present invention includes a method for replacing or reinforcing bone in vivo. The method comprises providing a viscous component and a plurality of biodegradable inclusions, wherein the inclusions comprise polymers. In addition, the method comprises combining the viscous component and the plurality of inclusions to produce the bone replacement material and applying the bone replacement material in vivo to replace or reinforce bone.

Other embodiments include the inclusions having a surface to volume ratio of greater than six times the unit edge length of the bounding box. Further embodiments include the inclusions having an aspect ratio greater than 1. In addition, suitable polymers include poly(l-lactic acid) [PLLA], poly(d l-lactic acid) [PDLLA], poly(glycolic acid) [PGA], poly(lactic-co-glycolic acid) [PLGA], poly(paradioxanone), poly(dl-glycolic acid) [PDLGA], poly(propylene fumarate) [PPF], oligo(PEG fumerate) [OPF], poly(ethyleneglycol) [PEG], poly(caprolactone) [PCA], poly(hydroxybutyrate) [PHB], poly(hydroxy valerate) [PHV], poly(SA-HDA anhydride), poly(orthoesters), poly(phosphazenes), and copolymers of di-lactic acid and dl-glycolic acid.

It will therefore be seen that a technical advantage of the present invention includes an improved bone replacement material and methods for making and using bone replacement materials that overcome the problem of poor biodegradation. Further advantages include vascularization and growth of new tissue in an interconnected porous network.

The foregoing has outlined rather broadly the features and technical advantages of the present invention in order that the detailed description of the invention that follows may be better understood. Additional features and advantages of the invention will be described hereinafter that form the subject of the claims of the invention. It should be appreciated by those skilled in the art that the conception and the specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims.

### BRIEF DESCRIPTION OF THE DRAWINGS

For a detailed description of the preferred embodiments of the present invention, reference will now be made to the accompanying drawings, in which:

FIG. 1 is a top view of a plurality of two-dimensional inclusions;

FIG. 2 is a top view of a plurality of three-dimensional inclusions; and

FIG. 3 is a section view of a bone replacement material comprising inclusions.

### DETAILED DESCRIPTION OF THE PREFERRED EMBODIMENTS

It has been discovered that a bone replacement material in the form of a composite material comprising at least one viscous component and a plurality of biodegradable inclusions is biocompatible with natural bone and also facilitates bone growth. The bone replacement material is capable of being rendered porous for tissue growth and also is suitable for replacement of load bearing bones. For instance, it has been discovered that the bone replacement material of the present invention creates new trabeculae in bone tissue and can increase bone mass. Such new trabeculae can result in an increase in trabeculae connectivity. The inclusions preferably have a high surface to volume ratio.

Viscous components are well known in the art, and the viscous components of the present invention can include any fluid suitable for being cured into a hardened material. For instance, viscous components are typically used in bone cements, and the viscous components of the present invention can comprise any such components. It is to be understood that a “fluid” is a continuous, amorphous substance that comprises molecules that move freely past one another and typically can also assume the shape of its container. Preferably, the viscous components are aqueous-based compositions or polymers. More preferably, the viscous components are polymers. The viscous components can be biodegradable or not biodegradable, preferably biodegradable.

Aqueous-based compositions for use as viscous components are well known in the art, and the aqueous-based composition of the present invention includes any aqueous-based composition suitable for use with the biodegradable inclusions. The aqueous-based composition preferably comprises an aqueous lubricant and at least one calcium source. The aqueous lubricant can comprise saline solution, drug solution, phosphate-buffered saline (PBS), and/or pure water, preferably pure water. Without limiting the present invention, examples of pure water include deionized water, distilled water, and the like. The amount of aqueous lubricant used depends on the desired consistency of the composite material. Calcium sources are well known in the art, and the calcium source of the present invention can comprise any suitable calcium source for forming the composite material. For instance, calcium sources are typically used in bone cements, and the calcium sources of the present invention can comprise any such sources. For example, suitable calcium sources include calcium phosphates, CaCO₃, CaOH, CaO, CaNO₃, CaCl₂, CaF₂, Ca alginates, hydroxyapatite (HA) and the like, preferably calcium phosphates. Calcium phosphates of the present invention comprise any calcium phosphates suitable for replacing bone. Preferable calcium phosphates include acidic calcium phosphate (such as monocalcium phosphate monohydrate, monocalcium phosphate anhydrous, dicalcium phosphate anhydrous, dicalcium phosphate dihydrate, and the like) and basic calcium phosphate (such as tetracalcium phosphate, tricalcium phosphate, and the like).

Polymers for use as viscous components are well known in the art, and the polymers of the present invention include any polymers suitable for use in replacing bone. For instance, polymers are typically used in bone cements, and the polymers of the present invention can comprise any such polymers used in bone cements. Without limiting the present invention, examples of suitable polymers include poly(l-lactic acid) [PLLA], poly(d l-lactic acid) [PDLLA], poly(glycolic acid) [PGA], poly(lactic-co-glycolic acid) [PLGA], poly(paradioxanone) [PDS], poly(dl-glycolic acid) [PDLGA], poly(propylene fumarate) [PPF], oligo (PEG fumerate) [OPF], poly(ethyleneglycol) [PEG], poly(caprolactone) [PCA], poly(hydroxybutyrate) [PHB], poly(hydroxy valerate) [PHV], poly (SA-HDA anhydride), poly(orthoesters), poly(phosphazenes), and copolymers of di-lactic acid and dl-glycolic acid. Preferable polymers include PLG, PLLA, PGA, and PCL.

The inclusions are biodegradable and preferably comprise sub-millimeter and/or micro-sized particles, preferably with a bounding box length less than 500 microns. It has been discovered that the biodegradable inclusions increase the overall mechanical strength of the bone replacement material. The inclusions comprise any biodegradable polymers that are suitable for use in in vivo implantation applications. Examples of such biodegradable polymers include those described by Kulkarni, et al., J. Biomedical Materials Research, 5, 169-81 (1971) and Hollinger, J. O. and G. C. Battistone, “Biodegradable Bone Repair Materials,” Clinical Orthopaedics and Related Research, 207, 290-305 (1986), which are incorporated herein by reference in their entirety to the extent that such references are not contrary to the teachings of the present invention. Preferable inclusions include poly(paradioxanone), poly(dl-lactic acid), poly(dl-glycolic acid), poly(propylene fumarate), oligo (PEG fumerate) and copolymers of dl-lactic acid and dl-glycolic acid, and any mixtures thereof. In a preferred embodiment, the viscous component and the inclusions comprise at least one of the same polymer. The polymers are preferably cross-linked. The polymers can be cross-linked by any methods suitable for achieving a desired amount of cross-linking. Preferable cross-linking methods include exposure to thermal, photo, and/or chemical agents. In alternative embodiments, the polymers are not cross-linked. The composite material can comprise any desired amount of inclusions, preferably the inclusions comprise more than 30 vol. % of the composite material, more preferably between 30 and 80 vol. % of the composite material, and most preferably between 50 and 60 vol. % of the composite material.

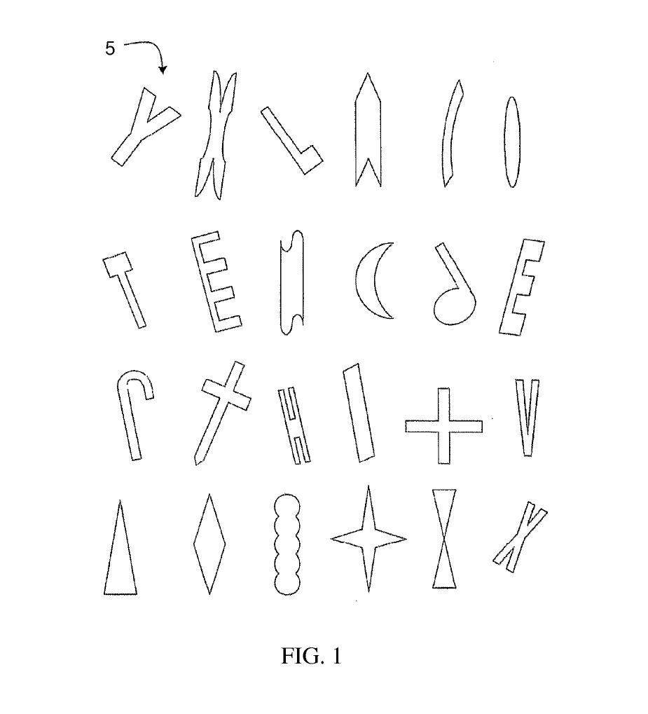

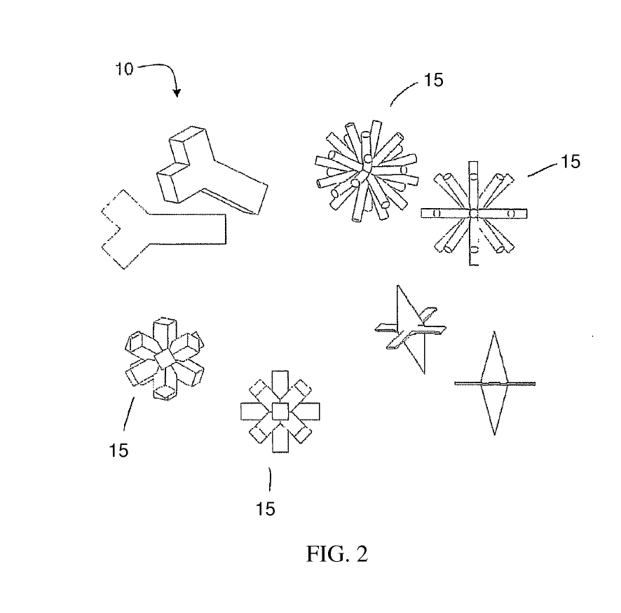

The inclusions can have any desired shape, preferably an engineered (pre-fabricated) shape. Such shapes can be two-dimensional or three-dimensional, with the inclusions comprising any desired two-dimensional or three-dimensional shape. FIG. 1 illustrates two-dimensional inclusions 5, and FIG. 2 illustrates three-dimensional inclusions 10. Preferably, the inclusions are three-dimensional. The preferable three-dimensional shapes are three-dimensional stars 15. It is to be understood that three-dimensional stars 15 are not limited to such illustrated shapes as shown in FIG. 2 but can include any suitable shape. When selecting a three-dimensional shape, it is preferable to select a shape that has a high surface to volume ratio. It is to be further understood that it is typically desirable for a bone replacement material to have a high permeability and a low porosity. Porosity is a volumetric measure of the void spaces within a porous solid. Permeability is a measure of the ease with which a liquid or gas flows through the porous solid. While encased pores can count towards porosity, they have no influence on permeability. Without being limited by theory, it is also to be understood that high permeability and low porosity are facilitated by a high surface to volume ratio of the degradable particles. Preferable surface to volume ratios are greater than six times the unit edge length of a bounding box, and more preferably 30 times greater. It is to be understood that the bounding box describes the overall dimensions that a particle occupies in terms of height, width, and depth if that particle would be placed in a square box. For instance, a cube having a side length “a” has a surface area to volume ratio of 6/a, and a sphere having a diameter “d” has a surface area to volume ratio of 6/d. Cubical and spherical inclusions can have a surface to volume ratio higher or lower than 6/a and 6/d, respectively. Preferably, such inclusions are higher than 6/a and 6/d, respectively.

It is also desirable for the inclusions to have a high aspect ratio. It is to be understood that an aspect ratio is the ratio of the major diameter to the minor diameter of an inclusion. It is also to be understood that high permeability and low porosity are facilitated by a high aspect ratio of the particles. Preferably, the inclusions have an aspect ratio greater than 1, more preferably greater than 2, and most preferably greater than 4. For instance, three-dimensional stars 15 have an aspect ratio of about 30. In addition, a three-dimensional star (not illustrated) that comes to points can have an aspect ratio that is higher than 30 and in some instances it can have an aspect ratio near infinite.

As noted, the inclusions preferably have an engineered shape. Methods for constructing the shape of sub-millimeter and micro-sized particles are well known in the art, and the inclusions of the present invention can be constructed by any method suitable for engineering such particles. Preferable methods for constructing the inclusions include stereo-lithography, photo-lithography, three-dimensional printing, fused depositioning, cutting, milling, extruding, stamping, separating inclusions from a material such as a block or sheet, and chemical applications such as controlled growth or self-assembly. More preferable methods include stereo-lithography, photo-lithography, stamping techniques, three-dimensional printing, extrusion, and fused deposition modeling.

The viscous components and the inclusions can be combined by any suitable method for forming the composite material. Preferably, the viscous components and the inclusions are mixed together to form the composite material. The viscous materials and the inclusions are sufficiently mixed for the composite material to take any desired form, preferably a flowable and paste-like form.

The bone replacement material can be applied to the body by an injection process or by a molding process. Preferably, the bone replacement material is applied to the body by the injection process. The injection process involves injecting the bone replacement material into the bone. The bone replacement material can be injected into the bone by syringe, a hand operated pump and the like, preferably a syringe. Preferably, the syringe is connected to a canula, which is already connected to the bone. The injection process can result in the viscous component and inclusions being further mixed. For instance, when the bone replacement material is injected into a bone marrow space, such mixing can result. The bone replacement material is preferably injected at a pressure sufficient to locally displace bone marrow without yielding the surrounding bone material. Without being limited by theory, it is believed that injection at such pressures allows the bone replacement material to flow between the micro-architectural structure of the bone with at least partial control. After injection, the bone replacement material is allowed to cure. Curing of bone replacement materials is well known in the art, and curing of the bone replacement material of the present invention can comprise any suitable curing process. For instance, processes for curing bone cements are well known in the art, and curing of the bone replacement materials can be accomplished by any such processes. For composite materials having viscous components comprising aqueous-based compositions, the bone replacement materials are cured by chemical applications such as cross-linking and/or physical applications such as curing. For composite materials having viscous components comprising polymers, the bone replacement materials are cured by thermal, chemical, and/or photo cross-linking agents. Examples of suitable thermal and chemical agents include benzoyl peroxide and n,n-demethyl-p-toluidine (DMT). An example of a suitable photo agent includes bis(2,4,6-trimethylbenzol)phenylphosphineoxide (BAPO).

The molding process comprises creating a mold of the implant to be implanted into the body. The molds can be created by any known process. For instance, the mold can be created using images of CT-scans and a rapid-prototyping machine. The mold is filled with the bone replacement material, and the bone replacement material is allowed to cure to form the mold. The bone replacement material mold is then implanted into the body after it is suitably cured.

The cured bone replacement material has a compressive strength that allows it to be suitable as a replacement and/or support for bone. The cured bone replacement material preferably has a compressive strength substantially the same as that of the surrounding bone. In alternative embodiments, the cured bone replacement material has a compressive strength within +200% of the surrounding bone. In other alternative embodiments, the cured bone replacement material has a compressive strength of at least about 20 MPa, preferably at least about 40 MPa and more preferably at least about 50 Mpa. Such cements have sufficient strength to withstand typical physiological and hyper-physiological stresses.

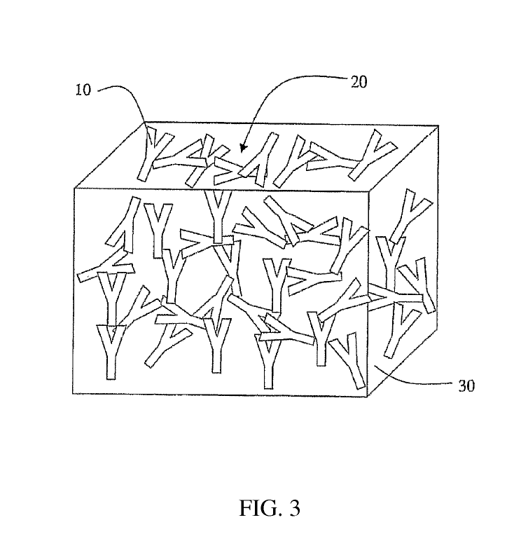

Preferably, the inclusions provide an interconnected “fibrous” network within the bone replacement material. It is also preferable to have all of the inclusions in contact with at least one other inclusion. It is to be understood that the invention is not limited to all of the inclusions having such contact but can also include less than all of the inclusions in contact with at least one other inclusion. FIG. 3 is an example of such an interconnected network 30. In FIG. 3, all of inclusions 10 are in contact with at least one other inclusion 10 in bone replacement material 20. Without being limited by theory, it is believed that the fibrous bone replacement materials behave similarly to other known fiber reinforced composites, such as fiberglass, where the random array of fibers strengthen the composite.

Biodegradation of the inclusions and the viscous component occurs at a predetermined or an undetermined rate, preferably a predetermined rate. The viscous components and inclusions can biodegrade at the same or different rates, preferably different rates. Many different mechanisms and factors affect the biodegradation. Mechanisms for biodegradation include surface erosion and bulk degradation. Surface erosion involves dissolving of the inclusions, with the dissolution beginning at the surface of the bone replacement material. Bulk degradation involves dissolution of the entire volume of the inclusions. Factors that affect the predetermined biodegradation rate include varying compositions of the inclusions, varying procedures for cross-linking of the polymers, adding solvents, adding inhibitors, increasing crystallinity of the polymer, strength of the cross-linking, and the like. Biodegradation of the inclusions creates pores in the bone replacement material. Without being limited by theory, it is believed that such pores allow vascularization and tissue growth within the bone replacement material. After biodegradation, the bone replacement material has between about 30 and 80 percent porosity. Preferably, the bone replacement material has less than about 70 percent porosity, more preferably more than about 40 percent porosity, and most preferably between 50 and 60 percent porosity. Biodegradation preferably occurs after curing of the bone replacement material. In alternative embodiments, the inclusions are physically removed from the bone replacement material after curing.

Further embodiments include the inclusions and/or the viscous component comprising therapeutic agents. Such therapeutic agents can be incorporated into the inclusions and/or the viscous component by any known process, preferably by mixing the agents, for instance, with the polymer or the aqueous-based compositions. In an embodiment wherein the viscous component is also biodegradable, the same or different therapeutic agents can be incorporated into the viscous component and the inclusions. In embodiments wherein the composite material precursor and the inclusions are biodegradable at different rates, such therapeutic agents are released at different times. Therapeutic agents are well known in the art, and the therapeutic agents of the present invention comprise any such agents suitable for release in the body. Examples of therapeutic agents include medicines, tissue growth promoters, chemotherapy agents, antibiotics, immune stimulators, immune suppressants, and the like. Examples of suitable tissue growth promoters include members of the transforming growth factor beta superfamily, bone morphogenic proteins, basic fibroblast growth factor, platelet derived growth factors, insulin-like growth factors, and extra cellular matrix molecules including osteopontin, osteonectin, osteocalcin, and bone sialoprotein. Preferably, the presence of tissue growth promoters encourages the growth of new tissue on or into the bone replacement material. In addition, the biodegradation rate can be predetermined so that the release time and dosage of the therapeutic agents meets the needs of the patient.

The bone replacement materials of the present invention can be used as bone replacement material in any suitable application or type of bone, including orthopaedic, dental and cranio-maxillofacial applications. For instance, the bone replacement material can be used in vertebroplasty and kyphoplasty applications, which relate to two minimally invasive procedures used to treat spinal compression fractures from osteoporosis, tumors, and trauma. The described techniques are not limited to fracture repair and prophylactic treatment. In vertebroplasty, under fluoroscopic guidance, a needle is placed into a compressed vertebra and bone replacement material is injected into the fracture. The result is that the vertebral fracture is stabilized, thereby restoring mechanical properties of the vertebra. In kyphoplasty, under fluoroscopic guidance, a needle is placed into the vertebra, then replaced with a balloon catheter. The balloon is positioned where, when inflated, it can affect the severe part of the fracture, usually the anterior portion. The balloon is inflated under sufficient pressure, and a space is created in the vertebra for the bone replacement material. The balloon catheter is removed, and the space is filled with bone replacement material. The procedure is repeated on the other side of the spine so that two balloon placements are done per vertebra. The result is that a balanced repair is created.

It is to be understood that the inclusions of the present invention are not limited to use in bone replacement material but instead can be used in any material where it is desirable to create an interconnected pore structure that does not significantly compromise the mechanical properties of the material. Examples of such materials include applications for drug delivery in soft tissue therapy such as in cancer treatment, cartilage repair, and engineering applications. Other alternative embodiments include the inclusions incorporating one or more lumens or hollow sections that may be filled with one or more materials chosen for their structural or therapeutic properties.

Although the present invention and its advantages have been described in detail, it should be understood that various changes, substitutions and alterations may be made herein without departing from the spirit and scope of the invention as defined by the appended claims.

What is claimed is:

1. A method for creating a bone replacement material, wherein the bone replacement material comprises a composite material, comprising:
(A) providing a viscous component;
(B) providing a plurality of biodegradable inclusions, wherein the inclusions comprise polymers; and
(C) combining the viscous component and the plurality of inclusions to produce the bone replacement material.

2. The method of claim 1, wherein the viscous component comprises an aqueous-based composition or a polymer.

3. The method of claim 2, wherein the polymer comprises at least one polymer selected from the group consisting of poly(l-lactic acid), poly(d l-lactic acid), poly(glycolic acid), poly(lactic-co-glycolic acid), poly(paradioxanone), poly(dl-glycolic acid), poly(propylene fumarate), oligo (PEG fumerate), poly(ethyleneglycol), poly(caprolactone), poly(hydroxybutyrate), poly(hydroxy valerate), poly(SA-HDA anhydride), poly(orthoesters), poly(phosphazenes), and copolymers of dl-lactic acid and dl-glycolic acid.

4. The method of claim 1, wherein the inclusions comprise at least one polymer selected from the group consisting of poly(paradioxanone), poly(dl-lactic acid), poly(dl-glycolic acid), poly(propylene fumarate), oligo (PEG fumerate), copolymers of dl-lactic acid and dl-glycolic acid, and mixtures thereof.

5. The method of claim 1, wherein step (B) further comprises constructing the inclusions.

6. The method of claim 5, wherein the inclusions are constructed by at least one technique selected from the group consisting of stereo-lithography, photo-lithography, stamping techniques, three-dimensional printing, extrusion, and fused deposition modeling.

7. The method of claim 1, further comprising:
(D) curing the bone replacement material.

8. The method of claim 7, further comprising
(E) removing the inclusions.

9. The method of claim 8, wherein the inclusions are removed by biodegradation.

10. The method of claim 7, wherein the bone replacement material has a compressive strength of at least about 20 MPa.

11. The method of claim 1 further comprising applying the bone replacement material in vivo to replace or reinforce bone.

12. The method of claim 11, wherein the bone replacement material is applied in vivo by injection.

13. The method of claim 12, wherein the bone replacement material is allowed to cure after injection.

14. The method of claim 11, wherein the bone replacement material is applied in vivo by a molding process.

15. The method of claim 1, wherein step (C) further comprises combining therapeutic agents with at least one of the inclusions or the viscous component.

16. The method of claim 11, wherein the bone is replaced in vertebroplasty and kyphoplasty applications.

17. The method of claim 1, wherein the bone replacement material has between about 30 and 80 percent porosity.

18. A method comprising:
(A) providing a viscous component;
(B) providing a plurality of biodegradable inclusions, wherein the inclusions have a surface to volume ratio of at least six times the unit edge length of a bounding box; and
(C) injecting the viscous component and biodegradable inclusions into bone.

19. The method of claim 18 wherein the inclusions have a aspect ratio of greater than 1.

20. The method of claim 18 wherein the inclusions have an engineered shape.
